(19) United States
(12) Patent Application Publication
(10) Pub. No.: US 2002/0022770 A1
(43) Pub. Date: Feb. 21, 2002

(54) Surgical retractor apparatus and method of its use

(76) Inventor: Mark K. Borsody, Chicago, IL (US)

Correspondence Address: MARK KLINGLER BORSODY, 5512 S. WOODLAWN AVE, SUITE 403, CHICAGO, IL, 60637, US

(21) Appl. No.: 09/835,787

(22) Filed: Mar. 29, 2001

Related U.S. Application Data
(60) Provisional application No. 60/193,390, filed on Mar. 31, 2000.

(51) Int. Cl. A61B 17/02

(52) U.S. Cl. 600/207

(57) ABSTRACT

The present invention provides a contact surface between the blade of a surgical retractor and an underlying tissue that directly reduces the injury of the tissue caused by the application of pressure during the retraction process. The embodiments of the present invention all use at least one inflatable chamber to form the contact surface, and the contact surface is then placed on the upper face of an otherwise plain surgical retractor blade. If a single inflatable chamber or interconnected group of inflatable chambers forms the retractor assembly's contact surface, it/they will be inflated and deflated in a cyclic manner to intermittently relieve the pressure applied to the underlying tissue. If a plurality of inflatable chambers or groups of inflatable chambers form the retractor assembly's contact surface, they will be inflated and deflated in an alternating manner so as to continually or regularly shift the sites of pressure that are applied to the underlying tissue, thereby preventing tissue injury from a prolonged, unremitting application of pressure. A system involving an inflation actuator, a switching mechanism, and various sensor devices is also described to illustrate a method of changing or shifting the retraction pressure across a tissue.

### CROSS-REFERENCE TO RELATED APPLICATIONS:

not applicable

### STATEMENT REGARDING FEDERALLY SPONSORED RESEARCH OR DEVELOPMENT:

not applicable

### REFERENCE TO A MICROFICHE APPENDIX:

not applicable

### BACKGROUND OF THE INVENTION

The purpose of retraction in surgical procedures is to create an approach to a diseased area of an organ that is obscured by healthy tissue and that can be exposed when the healthy tissue is displaced. Once the healthy tissue is sufficiently displaced, it must then be retained in the retracted position for the duration of the surgical procedure. Devices that are designed to move healthy tissue and maintain it in a retracted position are referred to as surgical retractors. Specifically, surgical retractors used during surgery on nervous tissues (e.g., brain, spinal cord, nerves) are referred to as neuro surgical retractors. Like other surgical retractors, neuro surgical retractors consist of a substantially flat blade fashioned from pliable metal that is coupled at its proximal end to a shaft; the shaft of the retractor in turn can be fixed into a stable frame that is anchored to the surgical table or to the patient's body. Used in such a manner the neurosurgical retractor is able to apply pressure to and thus displace the nervous tissue, and thereafter maintain it in the displaced position. Displacing the retracted tissue creates an artificial space (“operative field”) within which the surgeon can operate on the previously hidden, diseased tissue.

The design and method of use of existing neurosurgical retractors does not take into account the fragility and irreparability of nervous tissues. This is an issue of considerable clinical importance, since the process of retraction has been implicated in the permanent injury (a stroke) of the retracted nervous tissue in as many as 10% of all neurosurgical procedures [Rosenorn J. and Diemer N., 1985. Journal of Neurosurgery 63: 608-11; Yundt K. D. et al., 1997. Neurosurgery 40: 442-51]. Existing neurosurgical retractors are modeled after retractors used in surgery of the abdomen and thorax. These non-nervous tissues are relatively resistant to pressure and are able to heal well or even regenerate after injury; by contrast, nervous tissues—particularly brain and spinal cord—are exquisitely pressure-sensitive, do not regain function after injury, and have minimal regenerative ability. Commonly-used neurosurgical retractors have no modifications to reduce tissue injury and they are generally used in a manner that ignores the factors governing tissue injury, namely (a) the amount of pressure applied to the tissue and (b) the duration of time that pressure is applied to the tissue. Studies in animal models and in neurosurgical patients indicate that even mild pressure (10 mmHg) applied to the brain cannot be maintained for longer than 8 minutes without critically compromising the blood flow to the nervous tissue [Rosenorn J., 1989. Acta Neurologica Scandinavia Supplement 120: 1-30]. However, even routine neurosurgical procedures require the application of as much as 75 mmHg of retraction pressure for upwards 40 minutes without relief [Rosenorn J., 1985. Acta Neurochirugica 85: 17-22].

Periodic reduction of the retraction pressure has been shown to reduce the incidence and severity of nervous tissue injury in animals [Rosenorn J. and Diemer N., 1988. Acta Neurochirugica 93: 13-7], but the state-of-the-art of surgical retractors does not include designs that would accomplish this in clinical neurosurgery. Several modifications of surgical retractors have been proposed to indirectly limit the injury to the tissue undergoing retraction. These modified surgical retractors measure either (a) the pressure applied to the tissue by the retractor blade (U.S. Pat. Nos. 4,263,900; 5,201,325; 5,769,781), or (b) the blood flow to and/or metabolic activity of the tissue underneath the retractor blade (U.S. Pat. No. 4,945,896). But while these modified surgical retractors could detect the onset of nervous tissue injury during a neurosurgical procedure, they do not inherently act to reduce the extent of the injury. Instead, tissue injury would only be prevented if such devices signaled to the surgeon to reduce or release the pressure applied by the retractor; however, a significant reduction or release of the retraction pressure would compromise a neurosurgical procedure since it would collapse the already limited operative field within which the surgeon was performing the procedure. A preferred neurosurgical retractor would then involve modifications to reduce or release the retraction pressure on a tissue without allowing the tissue to significantly change its position with respect to the boundaries of the operative field.

### BRIEF DESCRIPTION OF THE INVENTION

The adjuncts to surgical retractors that constitute the present invention are designed to reduce the injury of tissues caused by the retraction process. While the present invention is most obviously applicable to use on nervous tissues, other pressure-sensitive tissues may similarly benefit from its use.

At sites on the retractor blade where it directly contacts the underlying tissue, the pressure that is applied to the tissue reduces local blood flow and this may result in ischemia and tissue death. The adjuncts to surgical retractors described herein involve an inflatable chamber or plurality of inflatable chambers coupled to or placed upon the upper face of a retractor blade (“contact surface”). To retract a tissue, the inflatable chamber(s) on the retractor blade are placed against the tissue and sufficient pressure is applied to the tissue so as to displace it. The pressure applied to the underlying tissue by the contact surface of inflatable chamber(s) can then be regularly changed or shifted by altering the degree or pattern of inflation of the inflatable chamber(s), and in doing so the tissue will suffer less injury because blood flow to the tissue will not be critically compromised.

In the simplest embodiment of the present invention, a single inflatable chamber forms the contact surface on an otherwise plain retractor blade. The single inflatable chamber is in fluid communication with the output source of an inflation actuator through an inflation conduit. After the retractor assembly's contact surface is placed against the tissue—thereby sandwiching the inflatable chamber between the retractor blade and tissue—the inflatable chamber is inflated and deflated in a cyclic manner by the inflation actuator. During periods when the inflatable chamber is deflated, there will be reduced pressure applied to the underlying tissue, allowing for improved blood flow to the tissue without significantly compromising the position of the retracted tissue. A variation of this design involves a plurality of inflatable chambers that are all connected to the output source of an inflation actuator by a common inflation conduit; such a contact surface would be used as described previously for a contact surface composed of a single inflatable chamber.

In a more preferred embodiment of the present invention, the retractor assembly's contact surface is formed from a plurality of inflatable chambers, each of which is connected to its own inflation conduit. One of the plurality of inflatable chambers or a subset of the totality of inflatable chambers are then inflated under the direction of a switching mechanism that can direct fluid flow from the output source of an inflation actuator to selected inflation conduits. While a single inflatable chamber or a subset of the totality of inflatable chambers are in the inflated state, the remaining inflatable chambers that are not in fluid communication with the inflation actuator are brought to or maintained in the deflated state. After predetermined criterions are satisfied by measurements that are either inherent to the switching mechanism's function (e.g., the passage of a certain period of time) or that are generated by sensor devices (e.g., the crossing a threshold of pressure, a decrease in blood flow in the retracted tissue), the switching mechanism redirects fluid communication with the inflation actuator to another inflatable chamber or subset of the totality of inflatable chambers that were theretofore deflated; meanwhile, the loss of fluid communication between the inflation actuator and the previously-inflated inflatable chamber or chambers causes them to deflate. This process inflates each inflatable chamber or subset of the totality of inflatable chambers in an alternating manner, and after all inflatable chambers or subsets of the totality of inflatable chambers have had their turn in the inflated state the process will repeat in a cyclic manner. A similar process could be employed with a contact surface formed from a plurality of inflatable chambers in which said chambers are subdivided into two or more interspersed groups so that each group is defined by the ultimate connection of all its member inflatable chambers to a common inflation conduit. A retractor in which the contact surface is comprised of a plurality of inflatable chambers and that is utilized as described above will regularly shift the pressure applied by the retraction process across the underlying tissue so that no part of the tissue is subjected to the injurious effects of prolonged pressure.

### BRIEF DESCRIPTION OF THE SEVERAL VIEWS OF THE DRAWING

The above and other objects, features, and advantages of the present invention will become apparent to those skilled in the art upon an examination of the following description and drawings.

FIG. 1 is a perspective view showing a contact surface formed from a single inflatable chamber that is coupled to the upper face of a plain retractor blade.

FIG. 1A is an enlarged, perspective view of the enclosed area of FIG. 1 showing the contact surface partially removed so as to demonstrate the coupling of the inflatable chamber to the upper face of a plain retractor blade by an adhesive layer.

FIG. 2 is an enlarged, perspective view similar to that of the enclosed area of FIG. 1 showing: a contact surface composed of a plurality of inflatable chambers that are arranged in a motif of three groups of parallel channels; the coupling of the contact surface to the upper face of a plain retractor blade; and the connection of the member inflatable chambers of each group to a common inflation conduit.

FIG. 2A is a cross-section view taken along line 7-7 of FIG. 2 that shows how sequential inflation of the individual groups of parallel channels changes the distribution of pressure along a tissue.

FIG. 3 is an enlarged, perspective view similar to that of the enclosed area of FIG. 1 showing: a contact surface formed from a motif of square-based inflatable chambers arranged into two groups; the coupling of the contact surface to the upper face of a plain retractor blade; and the connection of the member inflatable chambers of each group to a common inflation conduit.

FIG. 3A is a plan view of the contact surface shown in FIG. 3 with the upper surfaces removed in order to demonstrate the manner in which the non-contiguous chambers of a given group are all ultimately in fluid communication with a common inflation conduit.

FIG. 4 is a perspective view showing a contact surface formed from three inflatable chambers that are all connected to a common inflation conduit, and the coupling of the contact surface to the upper face of a pocket designed to fit over a plain retractor blade.

FIG. 5 is a schematic for regulating the use of a contact surface composed of three inflatable chambers arranged as parallel channels that can be separately connected into fluid communication with the output source of an inflation actuator by means of a switching mechanism, and the use of intrinsic and extrinsic sensor devices to regulate the switching mechanism and inflation actuator.

### DETAILED DESCRIPTION THE INVENTION

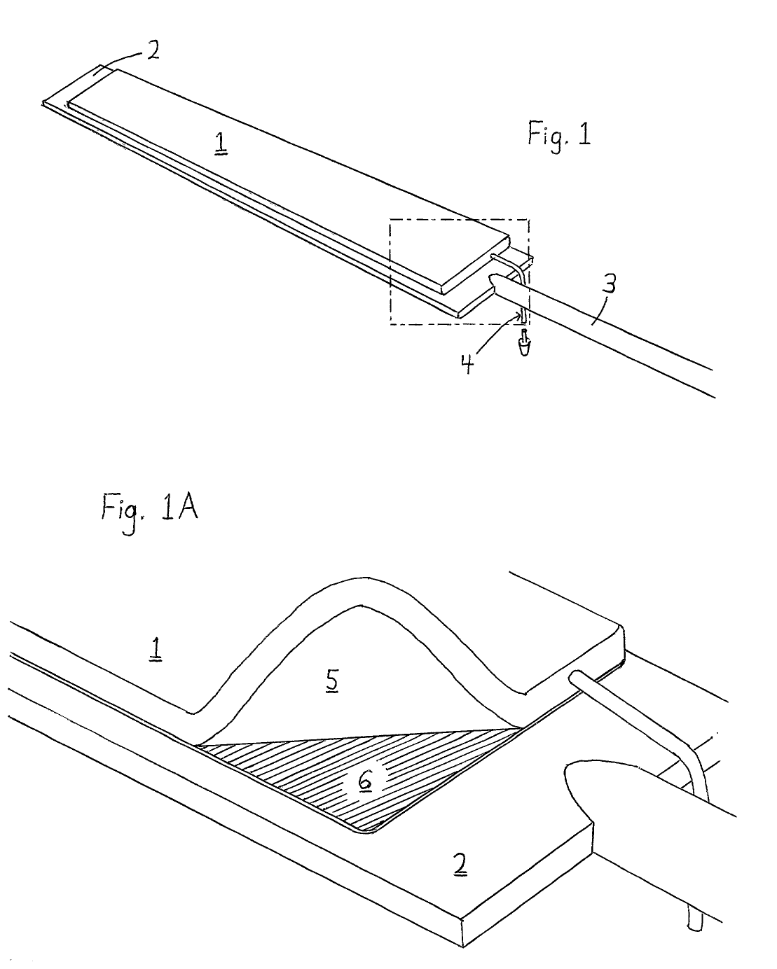

The present invention provides adjuncts for surgical retractors that will directly reduce the injury of tissue caused by the application of pressure during the retraction process. It is therefore an object of the present investigation to cover the upper face of a plain retractor blade that otherwise would directly contact the tissue with at least one balloon-like inflatable chamber. In the simplest embodiments of the invention, a single inflatable chamber or a plurality of inflatable chambers is coupled to the upper face of an otherwise plain retractor blade. The inflatable chamber(s) are ultimately connected by a single inflation conduit directly to the output source of an inflation actuator. The inflation actuator controls the degree of inflation of the inflatable chamber(s) by regulating fluid flow to and/or fluid pressure in the inflatable chamber(s). In order to retract tissue with the retractor assembly, the surface composed of an inflatable chamber or chambers is placed on the tissue rather than the plain retractor blade. The inflatable chambers form, then, a “contact surface” for the retractor blade. One of these simple embodiments of the present invention is shown in FIG. 1. A single inflatable chamber (1) is coupled by its lower face to the upper face of a plain retractor blade (2). A shaft (3) extends from the proximal end of the retractor blade and is intended for anchoring the retractor blade to a stable surgical frame or handle (not shown). The inflatable chamber is in fluid communication at its proximal end with an inflation conduit (4), and the inflation conduit is connected at its distal end to an inflation actuator (not shown) that controls the inflation and deflation of the inflatable chamber. In this example, the inflatable chamber will be cyclically inflated and deflated so that the pressure of retraction applied to the underlying tissue is continually or regularly changed. A contact surface formed from a plurality of inflatable chambers that are all connected to a common inflation conduit would be used in a similar manner. By relieving the pressure applied to the underlying tissue by the retractor assembly, a continuous and prolonged compression of the tissue that leads to tissue injury will not occur.

In more preferred embodiments of the present invention, the retractor assembly's contact surface is formed from a plurality of inflatable chambers that are not utilized as a single, coordinated group. Instead, each inflatable chamber can be in fluid communication with its own inflation conduit, or the plurality of inflatable chambers can be subdivided into groups so that all inflatable chambers of a given group are in fluid communication with a common inflation conduit. Because the plurality of inflatable chambers or groups of inflatable chambers will be inflated asynchronously, a switching mechanism must necessarily intervene between the output source of the inflation actuator and the plurality of inflation conduits. Said switching mechanism serves to direct fluid flow from the output source of the inflation actuator to a select inflation conduit or subgroup of the totality of inflation conduits. A contact surface formed from a plurality of inflatable chambers each with its own inflation conduit will be used in such a manner that only one inflatable chamber or subset of the totality of inflatable chambers are inflated at a time, while all other inflatable chambers are deflated. Similarly, if a plurality of inflatable chambers is subdivided into groups, only one group or subset of the totality of groups is inflated at a time while all other groups are deflated. By sequentially alternating the pattern of inflated and deflated inflatable chambers or groups thereof, the pressure applied to the underlying tissue will be regularly shifted, thereby avoiding a prolonged, static application of pressure that causes tissue injury.

Some of the aforementioned embodiments of a retractor assembly's contact surface involve a plurality of inflatable chambers. A plurality of inflatable chambers can be arranged in a near-infinite variety of ways, however the most practical and useful means of arranging the plurality of inflatable chambers is into a motif (i.e., a repetitive design). Some examples of simple motifs for contact surfaces formed from a plurality of inflatable chambers, and the method of their use, will now be explained.

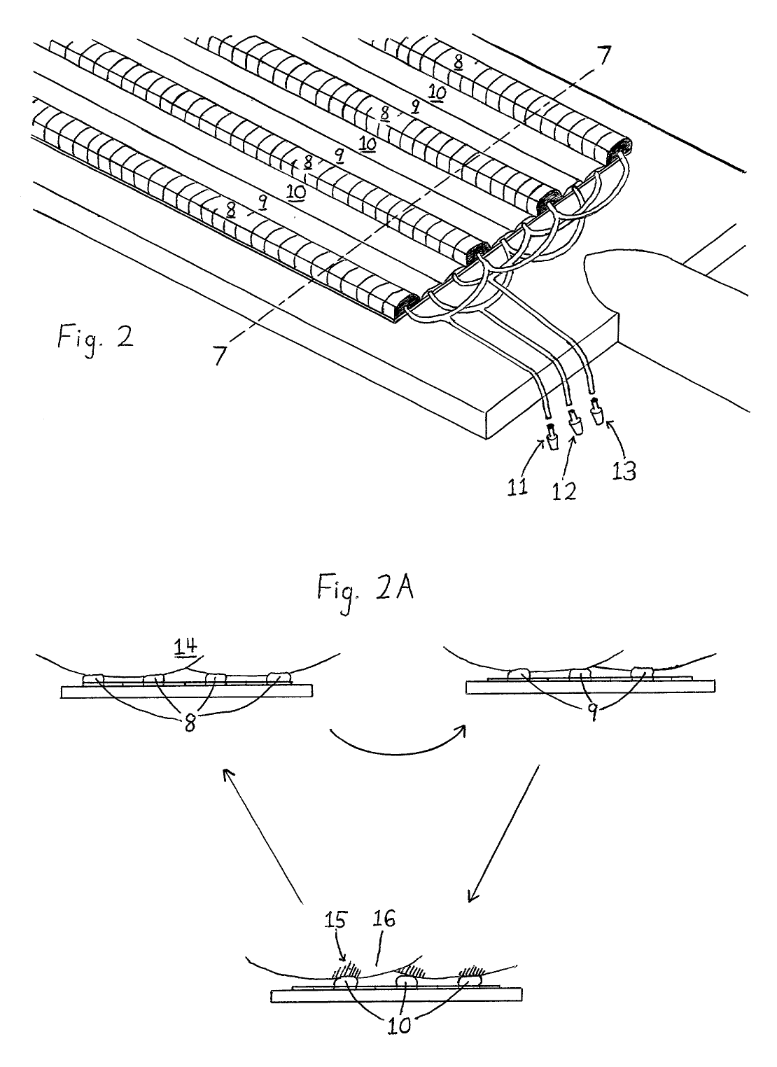

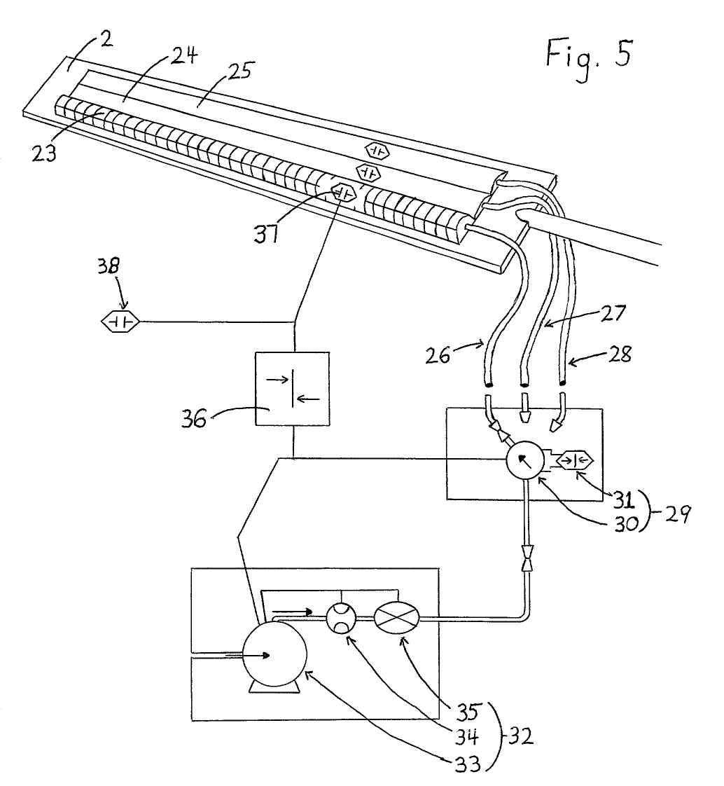

FIG. 2 depicts a contact surface formed from a plurality of inflatable chambers that are arranged as parallel channels. This drawing represents an enlargement of the left proximal corner of a retractor assembly, similar to the area outlined in FIG. 1. In this example, the inflatable chambers are subdivided into three interspersed groups of parallel channels (8, 9, 10), one group of which is shown in the inflated state (8) while the remaining two groups are shown in the deflated state (9, 10). All channels of a given group are connected at their proximal ends to a common inflation conduit; in this example, group (8) channels are connected to inflation conduit (11), group (9) channels are connected to inflation conduit (12), and group (10) channels are connected to inflation conduit (13). The inflation conduits can then be selectively placed into fluid communication with the output source of an inflation actuator by means of a switching mechanism (not shown; refer to FIG. 5). Thus, all the channels of a given group will be simultaneous inflated if and only if fluid flow is directed through the switching mechanism to the inflation conduit that supplies that group. FIG. 2A is a cross-section of the contact surface of the retractor assembly of FIG. 2 taken along line 7-7 that depicts the cyclic and progressive inflation of the various groups. This example demonstrates the shifting application of pressure along the tissue (14) by a retractor assembly's contact surface: the pressure initially applied by the channels of group (8) is shifted to group (9), and then finally to group (10) before returning back to group (8). In this arrangement of three groups of inflatable channels, there is contact with the tissue at regular intervals (15) that are separated by spaces of minimal contact where the channels are deflated (16). Since the three groups of channels can be alternately inflated by means of a switching mechanism (not shown; refer to FIG. 5), the contact surface formed from the inflatable chambers can be changed regularly or continuously to prevent prolonged compression of any particular portion of the tissue.

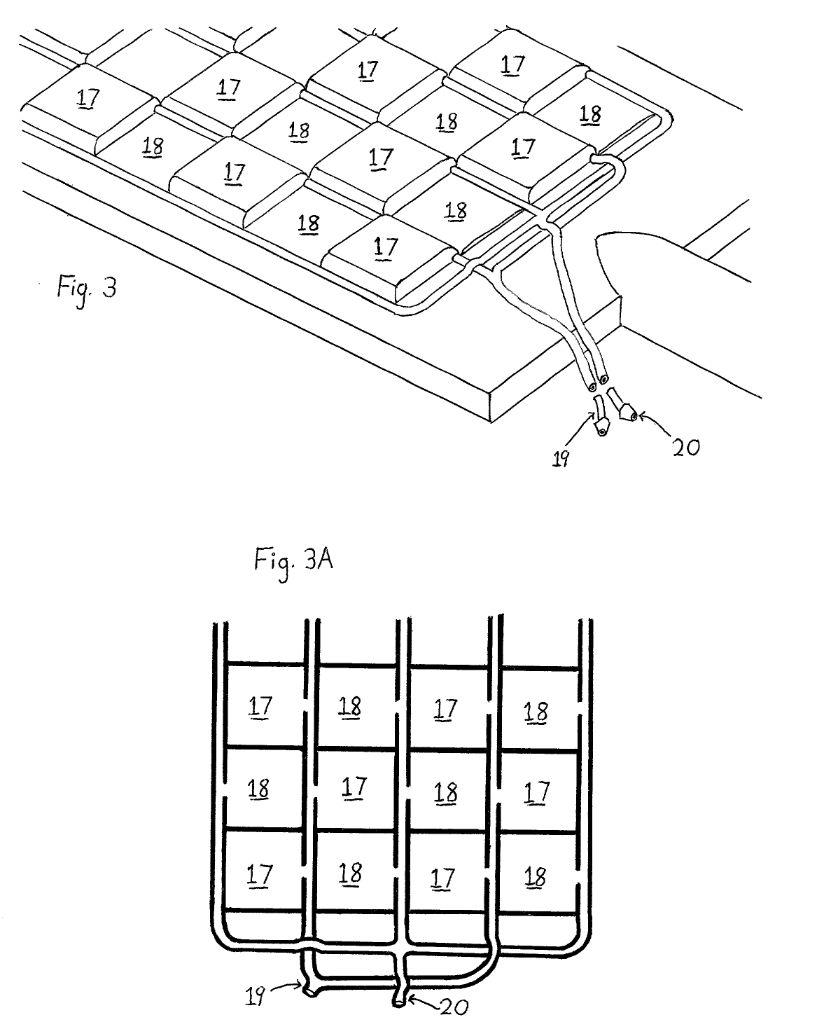

FIG. 3 depicts another contact surface formed from a plurality of inflatable chambers in which the bases of the inflatable chambers are square. This drawing represents an enlargement of the left proximal corner of a retractor assembly, similar to the area outlined in FIG. 1. The plurality of inflatable chambers are subdivided into two groups so that all member inflatable chambers of a given group are in fluid communication with a common inflation conduit: group (17) chambers are connected to inflation conduit (19), and group (18) chambers are connected to inflation conduit (20). In this example, the two groups of inflatable chambers are arranged so that there is a minimal juxtaposition of the member chambers of each group. One group of inflatable chambers is displayed in the inflated state (17), and the other group is displayed in the deflated state (18). In this embodiment of the invention, inflation of one of the two groups of inflatable chambers will distribute the pressure of retraction to the tissue in a checkerboard-like pattern. By means of a switching mechanism (not shown; refer to FIG. 5), the pressure of retraction can then be moved to non-compressed tissue by deflating the previously-inflated group of inflatable chambers and inflating the previously-deflated group of inflatable chambers. This process then repeats in a cyclic manner in order to continuously or regularly shift the pressure of retraction.

The contact surface described in FIG. 3 involves a motif of two groups of square-based chambers. Connecting all the member inflatable chambers of a given group together in fluid communication with a single inflation conduit necessitates a complex branching of the inflation conduit. FIG. 3A is a plan view of the motif shown in FIG. 3 that demonstrates the separate networks of fluid communication between the member chambers of each group (17, 18) that converge into the inflation conduits (19, 20). Such a contact surface would probably be best made from a single, large chamber that is subdivided internally with welds that define the boundaries of the individual members of the plurality of inflatable chambers, and the networks of fluid communication that converge into the inflation conduits.

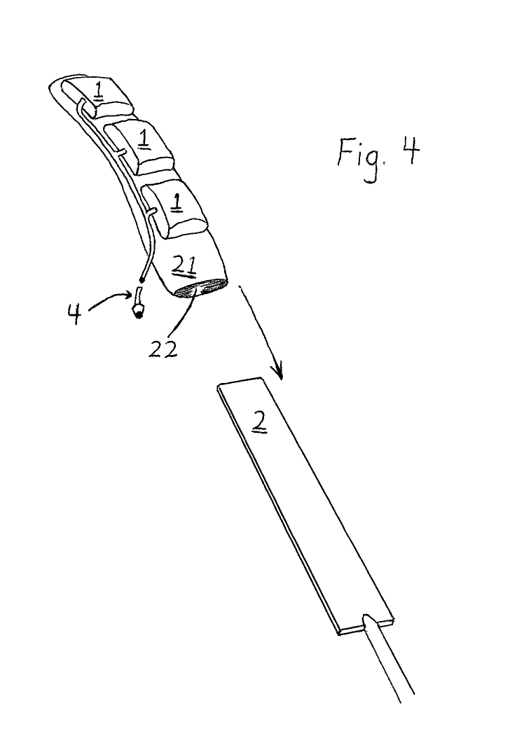

There are two means by which a contact surface formed from an inflatable chamber or chambers can be attached to a plain retractor blade: by direct adhesion of the contact surface to the retractor blade, and by joining the contact surface to a pocket that fits over the retractor blade. FIG. 1A demonstrates a method for directly attaching the contact surface to the upper face of a plain retractor blade. This drawing is an enlarged view of the left proximal corner of the retractor assembly shown in FIG. 1. In this embodiment of the present invention, the contact surface is formed from a single inflatable chamber that exhibits an upper face (1) and a lower face (5) separated by the inflatable chamber's lumen. The inflatable chamber is coupled by its lower face to the upper face of a plain retractor blade (2) by a layer of an adhesive material (6) applied to the lower face of the inflatable chamber. In another embodiment of the present invention, the inflatable chamber(s) that form the contact surface are coupled to, involved in, or formed from a pocket that can be pulled over a plain retractor blade. FIG. 4 shows an example of this embodiment in which the contact surface is formed from three inflatable chambers (1) that are all in fluid communication with a single inflation conduit (4). The inflatable chambers are coupled to the upper face of a pocket (21) that is closed on its distal end and lateral sides but is open on its proximal end (22); a plain retractor blade (2) can then be inserted into the open proximal end of the pocket so that the inflatable chambers rest on the blade's upper face. These two means of fixing a contact surface onto a plain retractor blade allow for the disassembly of the retractor blade and contact surface upon the completion of a surgical procedure. The separate parts can then be sterilized and reused in future procedures.

All of the embodiments of the present invention require for proper use an inflation actuator that is capable of regulating the volume of fluid flow to and/or the inflation pressure inside the inflatable chamber(s) that form the contact surface. Furthermore, some of the embodiments of the present invention require a switching mechanism that determines fluid communication between the inflation actuator and a select inflation conduit representing a single inflatable chamber or group of inflatable chambers, or a select subgroup of the totality of inflation conduits representing a plurality of inflatable chambers or groups of inflatable chambers. The simplest embodiments of the invention, in which a single inflation conduit is in fluid communication with a single inflatable chamber or all members of a plurality of inflatable chambers, do not require a switching mechanism because the inflatable chamber(s) are inflated and deflated in a cyclic manner that can be controlled entirely by the inflation actuator. In more preferred embodiments of the present invention, a plurality of inflatable chambers or groups thereof are inflated by the inflation actuator (i) in a sequential fashion if the array consists of more than two inflatable chambers or groups of inflatable chambers, or (ii) in an alternating fashion if the array consists of only two inflatable chambers or two groups of inflatable chambers. Each means of use will now be described for an example contact surface composed of a plurality of inflatable chamber divided into four groups of parallel channels A, B, C, and D, wherein the channels alternate - - - A-B-C-D-A-B-C-D - - - , etc., and wherein all channels of a given group are connected to a common inflation conduit. The four groups of inflatable chambers could be used in a sequential fashion as follows: starting from an initial state in which group A channels are in the inflated position and group B, C, and D channels are in the deflated position, the activation of the switching mechanism according to certain criterions would direct fluid flow toward group B channels, causing group B channels to inflate. The loss of fluid communication between the inflation actuator and group A channels would cause them to deflate; group C and D channels would meanwhile remain in the deflated position. The switching mechanism would progress to a state in which group C channels are inflated and group A, B, and D channels are deflated, and then to a state in which group D channels are inflated and group A, B, and C channels are deflated. The process would then revert to the initial state in which only group A channels are in the inflated position, and the cycle would repeat indefinitely. The four groups of parallel channels described above could also be utilized in an alternating fashion as follows: group A and C channels are simultaneously inflated via their respective inflation conduits while group B and D channels are in the deflated state. Then, by means of the switching mechanism, group B and D channels are inflated and group A and C channels are deflated. This process returns to the state in which group A and C channels are inflated and group B and D channels are deflated, and would repeat these steps in a cyclic manner.

The optimal control of the inflation actuator and switching mechanism would involve regulation of their functions by sensor devices that estimate directly or indirectly the viability of the tissue undergoing retraction. Intrinsic sensor devices (i.e., sensor devices within the inflation actuator or switching mechanism) may measure: fluid pressure at, or fluid flow from, the output source of the inflation actuator; or the duration of time that an inflation conduit or subgroup of inflation conduits are kept in fluid communication with the output source of the inflation actuator. Sensor devices that are extrinsic to the inflation actuator and switching mechanism may be located on the contact surface, on the retractor blade's upper face, or be placed separate from the retractor assembly, and they may measure any of the following: pressure, blood flow, metabolic activity, or electrical activity. Signals from extrinsic sensor devices would allow for the switching mechanism and inflation actuator to adjust and/or redistribute the pressure applied by the contact surface when dangerous amounts of pressure are being applied to the tissue. A representative system for using sensor devices in the control and coordination of the switching mechanism and inflation actuator is shown in FIG. 5. This example shows a simple array comprised of three inflatable chambers arranged as parallel channels (23, 24, 25) that are coupled to a plain retractor blade (2). Each of the three inflatable channels is connected by its individual inflation conduit (26, 27, 28) to a switching mechanism (29) that determines fluid communication with the output source of an inflation actuator (32). A variable switch (30) inside the switching mechanism has created fluid communication between the output source of the inflation actuator and a single inflation conduit (26), causing the inflation of one of the channels (23). The other channels (24, 25), which are not in fluid communication with the output source of the inflation actuator, are in their deflated states. Intrinsic sensor devices such as a timer (31) can initiate the redirection of fluid flow through the switch after the passage of a defined period of time. The activity of a pump (33) inside the inflation actuator and the fluid flow it creates may be regulated by intrinsic sensor devices such as a flow meter (34) or pressure gauge (35). Also, the function of the pump and switch may be modified by a comparator (36) that processes signals from extrinsic sensor devices placed on the contact surface (37) or that are independent of the retractor (38).

1. I claim a contact surface for surgical retractors that is comprised of:
a) an inflatable chamber;
b) the coupling of said inflatable chamber by its lower face to the upper face of a retractor blade;
c) an inflation conduit that is connected at its distal end into fluid communication with said inflatable chamber and that is connected at its proximal end into fluid communication with the output source of an inflation actuator.

2. The contact surface for surgical retractors of claim 1, wherein the inflation of said inflatable chamber is limited by internal adhesions or external appliances.

3. The contact surface for surgical retractors of claim 1, wherein at least one part of said inflatable chamber is a semipermeable membrane.

4. I claim a contact surface for surgical retractors that is comprised of:
a) a plurality of inflatable chambers;
b) the coupling of said plurality of inflatable chambers by their lower faces to the upper face of a retractor blade;
c) an inflation conduit or plurality of inflation conduits, wherein the proximal end(s) of said inflation conduit(s) (1) is/are connected directly into fluid communication with the output source of an inflation actuator, or (2) can be indirectly connected into fluid communication with the output source of an inflation actuator through an intervening switching mechanism.

5. The contact surface for surgical retractors of claim 4, wherein all inflatable chambers of said plurality of inflatable chambers are ultimately in fluid communication with the distal end of a common inflation conduit.

6. The contact surface for surgical retractors of claim 4, wherein each inflatable chamber of said plurality of inflatable chambers is in fluid communication with the distal end of an inflation conduit.

7. The contact surface for surgical retractors of claim 4, wherein said plurality of inflatable chambers is divided into groups so that all inflatable chambers of a given group are ultimately in fluid communication with the distal end of a common inflation conduit.

8. The contact surface for surgical retractors of claim 4, wherein said inflatable chambers are arranged in a motif.

9. The contact surface for surgical retractors of claim 4, wherein the inflation of said plurality of inflatable chambers is limited by internal adhesions or external appliances.

10. The contact surface for surgical retractors of claim 4, wherein at least one part of said inflatable chambers is a semipermeable membrane.

11. I claim an apparatus for covering a surgical retractor blade that is comprised of:
a) a pocket dimensioned to envelope said retractor blade, wherein said pocket is generally defined by an upper face, a lower face, a closed distal end, closed lateral edges, and an open proximal end so that said retractor blade can be inserted by its distal end into the open proximal end of said pocket;
b) an inflatable chamber that is coupled to, involved in, or formed from the upper face of said pocket so that said inflatable chamber forms the contact surface when said retractor blade is inserted into said pocket;
c) an inflation conduit that is connected at its distal end into fluid communication with said inflatable chamber and that is connected at its proximal end into fluid communication with the output source of an inflation actuator.

12. The apparatus of claim 11, wherein the inflation of said inflatable chamber is limited by internal adhesions or external appliances.

13. The apparatus of claim 11, wherein at least one part of said inflatable chamber is a semipermeable membrane.

14. I claim an apparatus for covering a surgical retractor blade that is comprised of:
a) a pocket dimensioned to envelope said retractor blade, wherein said pocket is generally defined by an upper face, a lower face, a closed distal end, closed lateral edges, and an open proximal end so that said retractor blade can be inserted by its distal end into the open proximal end of said pocket;
b) a plurality of inflatable chambers that are coupled to, involved in, or formed from the upper face of said pocket so that said plurality of inflatable chambers form the contact surface when said retractor blade is inserted into said pocket;
c) an inflation conduit or plurality of inflation conduits, wherein the proximal end(s) of said inflation conduit(s) (1) is/are connected directly into fluid communication with the output source of an inflation actuator, or (2) can be indirectly connected into fluid communication with the output source of an inflation actuator through an intervening switching mechanism.

15. The apparatus of claim 14, wherein all inflatable chambers of said plurality of inflatable chambers are ultimately in fluid communication with the distal end of a common inflation conduit.

16. The apparatus of claim 14, wherein each inflatable chamber of said plurality of inflatable chambers is in fluid communication with the distal end of an inflation conduit.

17. The apparatus of claim 14, wherein said plurality of inflatable chambers is divided into groups so that all inflatable chambers of a given group are in fluid communication with the distal end of a common inflation conduit.

18. The apparatus of claim 14, wherein said inflatable chambers are arranged in a motif.
